(19) United States
(12) Patent Application Publication
(10) Pub. No.: US 2003/0212006 A1
(43) Pub. Date: Nov. 13, 2003

(54) Method for reducing free radical formation in healthy individuals undergoing hypoxic exercise and medical conditions with increased oxygen free radicals

(76) Inventors: John G. Seifert, St. Cloud, MN (US); Linda M. Shecterle, Minneapolis, MN (US)

Correspondence Address: Linda M Shecterle, 940 Fernbrook Lane North, Minneapolis, MN, 55447, US

(21) Appl. No.: 10/144,318

(22) Filed: May 13, 2002

(51) Int. Cl. A61K 31/70; A61K 31/4162; A61K 31/198; A61K 31/7076; A61K 38/43

(52) U.S. Cl. 514/23; 514/47; 514/386; 514/563; 424/94.1

(57) ABSTRACT

A method of inhibiting the formation of oxygen-containing free radicals and derivatives of them during and following exercise is disclosed. It includes administering an amount of a supplement containing one or more compounds selected from the group consisting of pentose carbohydrates and derivatives thereof in conjunction with said exercise. The supplement is administered as a dose at one or more times selected from prior to, during and after exercise.

### FIELD OF INVENTION

The present invention relates to reducing free radical formation, which can occur during normoxia (sufficient levels of oxygen), hypoxia (insufficient or low levels of oxygen), hyperoxia (high levels of oxygen), changes in cellular and/or systemic temperature (such as hyperthermia or hypothermia), changes in cellular and/or systemic pH (such as acidosis), and/or changes in phosphate, glycolytic, or aerobic (oxidative phosphorylation) metabolism in cellular or systemic responses when the supplementation of pentose sugars (such as ribose, xylose, xylulose), alone or in combination with other macronutrients (such as carbohydrates, fats, proteins or derivatives thereof), electrolytes, enzymes, enzymatic inhibitors, organic and/or inorganic substances, antioxidants, and/or micro nutrients is utilized. The supplementation can be given orally, intravenously, intraperitoneally, or transdermally before, during, or after specific onset of physiological conditions or disease states in order to attenuate or prevent free radical formation. Whereas the attenuation and/or prevention of free radical formation could enhance a physiological condition and/or prevent or stabilize a physiological disease state and/or prevent or stabilize a physiological deterioration; or can aid in the prevention of symptoms in physiological conditions and/or disease states.

### BACKGROUND OF THE INVENTION/RELATED ART

In mammals, perpetual metabolic reactions continuously produce an array of biochemical compounds. One group of compounds produced through metabolic reactions is oxygen free radicals (a.k.a. reactive oxygen species, superoxides, peroxides, oxidative radicals, hydroxyl radicals). For example, hydrogen peroxide is produced when adenosine is catabolized to uric acid. Oxygen free radicals are compounds that have at least one unpaired electron in the outer orbital. This unpaired electron makes the free radical compound highly unstable and reactive with other molecules. This reactivity may alter numerous cellular functions or reactions within each in vivo cell or between cells, tissue beds, organs, bodily systems, or in in vitro tissue, such as preserved organs or tissue components as blood products, all of which will ultimately compromise the cell's or tissue's physiological function or potentially survival.

Free radical(s) reactions occur in three steps with, possibly, multiple substeps (Bus, 1979). The first reaction is the generation of the free radical, such as superoxide anion (O₂⁻), hydrogen peroxide (H₂O₂), or the hydroxyl radical (OH⁻). The second step is the propagation phase in which a new free radical is produced. Thereafter, the free radical may bind quickly to other molecules setting off another series of reactions, which can be damaging to the original molecule. The third step is the final phase where there is destruction of the free radical by the production of a stable compound.

Normal, Healthy Individuals

Free radicals are produced under both normal and abnormal conditions of metabolism, nutritional stress, physiological stress, altered environmental oxygen concentrations (including high altitude and deep water submersion), radiation, and chemicals or chemical reactions. Free radicals are also formed when oxygen is shuttled through the mitochondria in the electron transport chain during aerobic metabolism. Physiological conditions, such as changes in cellular and/or systemic temperature, changes in cellular and/or systemic pH may also lead to an increased production of free radicals. Tissue hypoxia or an ischemia/reperfusion state have been associated with the production of reactive oxygen species (oxygen free radicals). Ischemia/re-perfusion is a condition of inadequate blood supply to a tissue bed, followed by adequate or above normal blood flow to the affected tissue. Hypoxia has also resulted in both oxidant formation and oxidative stress agents (Park and Kehrer). Furthermore, the production of some oxygen free radicals increase during and following exercise, while other free radicals may reach peak concentrations within 12 to 24 hours post exercise.

Medical Conditions

Free oxygen radical production has been implicated in numerous altered physiological states and ailments. These produced free radicals can alter protein and deoxynucleic acid (DNA) structures, affect cancer and myocardial infarction rates, change cell membrane stability (lipids integrity), and prolong recovery time (Saladin).

An example of the production of oxygen free radicals can be found in patients with congestive heart failure (CHF). Elevated levels of malondialdehyde (MDA) have been measured in the serum of patients with CIE; and furthermore, these levels increase as one's failure becomes worse, as judged by one's potential progression in the New York Heart Association (NYHA) classifications (Serdar). Serdar et al concluded that oxidative stress with the production of oxygen free radicals increases systemically in patients with CHF and this increase correlates with the NYHA heart failure functional classes.

Implications for free radicals as major contributors to ischemic and excitotoxic tissue injury in the CNS has been described by Dubrinsky et al. Additionally, decreases in brain metabolism due to aging and/or neurodegeneration have been discussed by Jenkins et al. Brain cells are dependent on a permanent supply of high-energy phosphate compounds to fuel membrane ionic pumps, including ATP dependent enzymes and subsequent function. Potentially a defective mitochondrial energy generating system, by means of excitotoxicity or other environmental conditions, can lead to either primary or secondary dysfunction, which can progress to ultimate neuronal death.

Studies involving Huntington's Disease revealed diminished cerebral metabolic rates for glucose metabolism and fluordeoxyglucose positron emission tomography scans demonstrated strong evidence for an impairment in tissue energy metabolism. Similarly, Alzheimer's Dementia has been shown to be associated with a low metabolic rate for glucose, especially in the effected areas of the brain. This altered glucose metabolic state, affects energy production and correlated function. Further, other neurological conditions have been associated with similar energy deficiencies. In summary, defects in oxidative/energy metabolism may lead to death or dysfunction of neurons. Therefore, the invention disclosed within, relating to the supplementation of ribose, other pentoses, their derivatives alone, and in combination with other ingredients appears to be essential in the treatment of neurologic disorders.

### SUMMARY OF THE INVENTION

The present invention relates to methods and compositions for inhibiting and/or reducing the formation of oxygen free radicals and their derivatives in healthy individuals and patients afflicted with medical conditions. The present invention provides ribose, other pentose sugars, or their derivatives alone, or in combination with other carbohydrates, electrolytes, minerals, enzymes, proteins, free fatty acids, free fatty acid carriers, micronutrients, macronutrients, or other ingredients designed to inhibit and/or reduce the production or existing state of oxygen free radicals or their derivatives.

### Description of Tables

Table 1 reports the mean percent change in urinary MDA levels at time intervals in subjects following a single bout of exercise under hypoxic conditions. Table 2 represents reduced mean plasma glutathione (GSH) levels at time intervals in subjects following a single exercise under hypoxic conditions. Table 3 reports the mean plasma uric acid levels at time intervals in subjects undergoing a single exercise bout under hypoxic conditions. Table 4 reports the percent change in heart rate at time intervals in subjects following a single exercise bout under hypoxic conditions. Table 5 represents the mean percent change in urinary MDA levels at time intervals in a single subject undergoing multiple bouts of exercise under hypoxic conditions. Table 6 reports the absolute heart rate at time intervals in a single subject undergoing multiple bouts of exercise under hypoxic conditions.

### DETAILED DESCRIPTION OF THE INVENTION

Oxygen free radicals are continuously produced by all in vivo cells/tissue beds and in preserved in vitro cells/tissues. Obviously, the production of these radicals plays an adverse role in each cell's/tissue/organ subsequent function, and programmed cell death, apoptosis. This invention addresses this adverse state, in that the use of pentose sugars or their derivatives alone, or in combination with other agents, such as other carbohydrates, macronutrients, electrolytes, enzymes, enzymatic inhibitors, organic and/or inorganic substances, and/or micronutrients, both in healthy subjects and in patients afflicted with disease conditions. Pentose sugars or their derivatives are the dominant component in proposed products, directed at reducing or establishing a state of maintenance of oxygen free radicals.

Ribose is a five-carbon carbohydrate. The term ribose is used to include D-ribose, the precursors, and other pentose carbohydrates and all derivatives. It is an essential component of nucleotides (i.e. adenosine) and DNA. Ribose has been found to aid in the production of adenine nucleotides following myocardial and skeletal ischemia and/or hypoxia (Pauly ref). Ribose enters the pentose phosphate pathway and is phosphorylated to ribose-5-phosphate. Further reactions generate phosphorylribosyl-1-pyrophosphate (PRPP); and thereafter, may produce purine or pyrimidine nucleotides. The activity of the pentose phosphate pathway in heart and skeletal muscle reveals that there are two rate-limiting enzymatic steps, glucose-6-phosphate dehydrogenase and 6-phosphogluconate dehydrogenase. Exogenous ribose bypasses these steps, providing the ability to enhance the production of nucleotides faster than the normal process.

The pentose phosphate and glycolytic pathways are linked together by the transketolase and transaldolase enzymes. Cells may synthesize additional adenosine triphosphate (ATP) when substrate enters the glycolytic pathway at the glyceraldehyde-3-phosphate step. Adenosine triphosphate synthesis may also continue through the aerobic energy system where ATP is produced in the mitochondria with the presence of oxygen. Energy from the catabolism of ATP is constantly required for a variety of cellular or systemic processes, including its integrity and function. This turnover and production of high energy compounds, i.e. ATP, is continuously being replenished. However, conditions arise, such as ischemia and/or hypoxia, in which the production of energy compounds is suppressed and may take a considerable amount of time for their recovery, if at all possible. Supplementation of ribose as a substrate reflects that nucleotide catabolism may be attenuated. Consequently, subsequent production of free radicals may also be reduced.

There is difficulty in measuring free radicals directly; and therefore, other indices of free radical production are measured. The difficulty in free radical measurement stems from the fact that free radicals react so quickly that they are often changed within seconds of production. Secondly, the measurement assays, to date, have a large degree of variability or are unreliable. Researchers believe that the measurement of MDA levels can identify an oxidative stress state, reflecting a state of cellular/tissue compromise, and of equal importance the trends in following these levels can be used to judge stability or worsening of the affected tissue. Plasma MDA levels have been shown to be less reliable than urinary levels for identification and trend surveillance. Therefore, measuring urinary MDA as a marker of free radical production appears to be a current standard of oxidative stress assessment (Janero).

It should be noted that the following dosages of ribose, other pentoses, or their derivatives would function adequately in this invention. The range of these pentose sugars will vary with the indication, (i.e. healthy subjects exercise, disease states), however, levels between 0.5 to 20 grams/dose, with an ideal 2 to 10 grams/dose appears to have the greatest benefit. Given the present disclosure, it should be assumed that many changes may be made in regards to specific ingredients and still obtain similar results without departing from the scope of the invention or the principles from which it was found.

The following examples are included to demonstrate the preferred embodiment of the invention. D-ribose is the preferred embodiment, however, to those skilled in the art it is known that certain pentose carbohydrates, such as xylitol and ribulose, are readily converted to D-ribose in vivo. Therefore, the term “ribose” is intended to include D-ribose and such precursors thereof and other pentose sugars and derivatives. It should be appreciated by those skilled in the art that the methods and dosages in the examples that follow represent methods and dosages discovered by the inventors to function well in the practice of this invention, and thus can be considered to constitute preferred modes for its practice. However, those skilled in the art should, in light of the present disclosure, appreciate that many changes can be made in the specific embodiments that are disclosed and still obtain a like or similar result without departing from the concept and scope of the invention. All such changes are considered to be within the spirit, scope, and concept of the invention as defined by the appended claims.

### EXAMPLE 1

Seven healthy, moderately to highly conditioned subjects volunteered to participate in this randomized, blinded, crossover trial. All subjects were between the ages of 23 and 41 years of age, and each subject cycled in two distinct trial arms, a ribose phase and a control phase. One week separated each trial arm. Subjects ingested either 7 grams of ribose in 250 mL of water or just plain water, five minutes prior to the start of exercise and again within five minutes following exercise.

The scheduled exercise was 25 minutes of cycling at a workload relating to the lactic acid threshold. Subjects inhaled a hypoxic gas mixture of 16% oxygen and 84% nitrogen through a one-way breathing valve during the exercise bout. Following the cycling exercise, subjects sat quietly for 60 minutes and breathed room air during the recovery phase. All tests were conducted at room temperature.

Blood samples from an indwelling catheter in a forearm vein were collected pre-exercise, at 8, 16, and 24 minutes of exercise, and 30 and 60 minutes post-exercise. A whole blood sample was analyzed for hemoglobin. All remaining samples were centrifuged for 15 minutes, and thereafter the plasma was pipetted into separate vials for storage. Urine was also collected pre-exercise, 30 minutes post-exercise, and 60 minutes post-exercise. All blood and urine samples were immediately frozen and stored at −70° C. Each frozen plasma sample was analyzed for total protein and uric acid levels. Plasma samples were also analyzed for reduced glutathione levels (GSH kit, Oxis Research, Portland, Oreg.) and volumes were corrected for potential fluid loss by measuring total protein values. The frozen urine samples were analyzed for MDA (MDA kit, Oxis Research, Portland, Oreg.). Urinary creatinine levels were used to correct MDA levels for any changes in renal clearance and volumes.

Analyzed urinary and plasma parameters are represented in Table 1. Percent change in urine MDA was significantly lower (p=0.01) following ribose ingestion compared to the placebo (n=7 subjects). Percent change in plasma GSH was significantly lower (p=0.000) following ribose ingestion (n=7 subjects) is represented Table 2. Percent change in plasma uric acid was similar between treatments (n=7 subjects, p>0.05) is represented in Table 3.

The cardiovascular parameters of heart rate and oxygen pulse saturation (SpO₂) were collected at 9 minutes, 17 minutes, and 25 minutes of exercise. Oxygen pulse saturation was collected with a Nonin pulse oximeter (Nonin Medical Inc., Plymouth, Minn.). Percent change in heart rate during exercise was significantly less (p=0.03) during the ribose supplementation than for the placebo (n=7 subjects, Table 4). The 4.8% vs. 7.5% represents heart rates of 175 beats per minute (bpm) and 181 bpm for ribose and placebo (n=7 subjects).

Subjectively, 6 out of 7 of the blinded subjects ranked the exercise easier and felt they had a faster recovery period after cycling when they ingested ribose. Comments and symptoms observed during recovery included decreased lethargy when ribose was ingested and subjects were much more talkative and alert during recovery when ribose was ingested. During the control trial, subjects expressed strong desires to take a nap during the recovery period. Additionally, four of the subjects reported that after leaving the laboratory, they went home and took long (>2 hours) naps.

### EXAMPLE 2

One subject underwent a repetitive, three day consecutive exercise schedule. The subject performed this exercise schedule twice. One session the subject received 7 grams of ribose in 250 mL of water and the other session the subject consumed just plain water. The ribose or plain water was given 5 minutes prior to the start and immediately following exercise. Each exercise bout was separated by one week. Each session of cycling lasted for 25 minutes with a workload corresponding to a determined lactic acid threshold. During each exercise bout, the subject inhaled a hypoxic gas mixture of 16% oxygen and 84% nitrogen through a one-way breathing valve. Following the cycling exercise, the subject sat quietly for 60 minutes and breathed room air during this recovery phase. All tests were conducted at room temperature.

Blood samples were collected from a forearm vein at pre-exercise, 8, 16, and 24 minutes of exercise, and 30 and 60 minutes post-exercise. A whole blood sample was analyzed for hemoglobin The remaining blood was centrifuged for 15 minutes with the obtained plasma pipetted into storage vials. Samples were analyzed for total protein and uric acid levels. Urine was collected pre-exercise, 30 minutes post-exercise, and 60 minutes post-exercise. All blood and urine samples were immediately frozen and stored at −70° C. Urine samples were analyzed for MDA (MDA kit, Oxis Research, Portland, Oreg.) and urine creatinine levels were measured in order to correct MDA for changes in renal clearance rates and volumes.

Cardiovascular parameters of heart rate and oxygen pulse saturation (SpO₂) were collected at 9 minutes, 17 minutes, and 25 minutes of exercise. Oxygen pulse saturation was collected with a Nonin pulse oximeter.

Urinary MDA measurements and physiological tabulation of heart rate during every day session using both test articles are represented in Tables 5 & 6.

We claim:

1. A method of inhibiting the formation of oxygen-containing free radicals and derivatives thereof during and following exercise comprising steps of:
a. Administering an amount of a supplement containing one or more compounds selected from the group consisting of pentose carbohydrates and derivatives thereof in conjunction with said exercise;
b. Wherein said supplement is administered as a dose at one or more times selected from the group of times consisting of prior to, during and after exercise.

2. A method as in claim 1 wherein said exercise induces a hypoxic condition in an exerciser.

3. A method as in claim 1 wherein said supplement contains additional metabolic compounds.

4. A method as in claim 3 wherein said additional metabolic compounds are selected from the group consisting of L-carnitine, CoQ10, fructose, 1,6 diphosphate (FDP), catabolic adenine triphosphate substrates, creatine, vitamins, and antioxidants/scavengers.

5. A method as in claim 1 wherein said supplement is administered by a method selected from the group consisting of oral, intravenous, intraperitoneal, transdermal, and subcutaneous.

6. A method as in claim 1 wherein said supplement is administered in doses containing from about 0.5 to 20 grams pentose sugars per dose.

7. A method as in claim 6 wherein said doses contain from about 2 to 10 grams of pentose sugars.

8. A method as in claim 4 wherein said supplement is administered in doses containing from about 0.5 to 20 grams pentose per dose.

9. A method as in claim 1 wherein said supplement is administered as doses prior to, during and following exercise and wherein said exercising is under hypoxic conditions.

10. A method as in claim 6 wherein said supplement is administered as doses prior to, during and following exercise and wherein said exercising is under hypoxic conditions.

11. A method as in claim 8 wherein said supplement is administered as doses prior to, during and following exercise and wherein said exercising is under hypoxic conditions.

12. A method of improving cardiovascular parameters of an exercise during and following exercise comprising steps of:
a. Administering an amount of a supplement containing one or more compounds selected from the group consisting of pentose carbohydrates and derivatives thereof in conjunction with said exercise wherein a hypoxic condition exists in the exerciser;
b. Wherein said supplement is administered as a dose at one or more times selected from the group of times consisting of prior to, during and after exercise; and
c. Wherein the administration amount and timing of administration of said supplement is such that it improves one or more cardiovascular parameters selected from heart rate, blood pressure and tissue oxygen delivery.

13. A method as in claim 12 wherein the amount and timing of administration of doses of said supplement is such that it reduces one or more symptoms selected from the group consisting of extended degrees of drowsiness, cramping of muscles, soreness of muscles and manifested systemic symptoms, such as nausea, vomiting and headaches resulting from exercising under hypoxic conditions.

14. A method as in claim 12 wherein the amount and timing of administration of doses of said supplement maintain or improve cardiovascular parameters, such as heart rate, blood pressure, tissue oxygen delivery when used during and following exercise in hypoxic conditions.

15. A method as in claim 12 wherein benefits are realized with a state of hypoxia, as low as 10-16% O2.

16. A method as in claim 13 wherein benefits are realized with a state of hypoxia, as low as 10-16% O2.

17. A method as in claim 14 wherein benefits are realized with a state of hypoxia, as low as 10-16% O2.

18. A method as in claim 12 wherein said improvement includes enhancing recovery from an extreme tissue energy deficient and physiological dysfunctional state following exercise under hypoxic state creating conditions.

19. A method as in claim 12 wherein said supplement contains additional metabolic compounds.

20. A method as in claim 1 wherein said group of pentose carbohydratres includes ribose, xylose, and xylulose.

21. A method as in claim 12 wherein said group of pentose carbohydrates includes ribose, xylose, and xylulose.
TABLE 1
PERCENT CHANGE IN URINARY MDA LEVELS*

| Test Article | 0 min | 55 min | 85 min |
|---|---|---|---|
| Ribose | 0 | 2.47 ± 9.1% | −1.83 ± 9.1% |
| Placebo | 0 | 22.2 ± 9.1% | 38.25 ± 9.1% |

*Percent change results reported as a mean ± standard deviation.
TABLE 2
PERCENT CHANGE IN PLASMA GSH LEVELS*

| Test Article | 0 min | 8 min | 16 min | 24 min | 55 min | 85 min |
|---|---|---|---|---|---|---|
| Ribose | 0 | 4.1 ± 2.8 | 0.73 ± 2.8 | 2.0 ± 2.8 | 0.04 ± 2.8 | −4.6 ± 2.8 |
| Placebo | 0 | 14.4 ± 2.8 | 11.5 ± 2.8 | 10.7 ± 2.8 | 3.3 ± 2.8 | 4.2 ± 2.8 |

*Results reported as a mean ± standard deviation
TABLE 3
PERCENT CHANGE IN PLASMA URIC ACID*

| Test Article | 0 min | 8 min | 16 min | 24 min | 55 min | 85 min |
|---|---|---|---|---|---|---|
| Ribose | 0 | 3.2 ± 2.8 | 9.3 ± 2.8 | 8.9 ± 2.8 | 9.9 ± 2.8 | 17.9 ± 2.8 |
| Placebo | 0 | 5.4 ± 2.8 | 8.7 ± 2.8 | 9.4 ± 2.8 | 12.8 ± 2.8 | 11.5 ± 2.8 |

*Results reported as mean ± standard deviation
TABLE 4
PERCENT CHANGE IN HEART RATE*

| Treatment | 9 min | 17 min | 25 min |
|---|---|---|---|
| Ribose | 0 | 3.5 ± 0.5% | 4.8 ± 0.5% |
| Placebo | 0 | 3.7 ± 0.5% | 7.5 ± 0.5% |

*Results reported as percent change ± standard deviation
TABLE 5
PERCENT CHANGE IN URINARY MDA LEVELS

| Test Article | Day | Time | % Change MDA |
|---|---|---|---|
| Ribose | 1 | 0 | 0 |
| | 1 | 55 | −12.9 |
| | 1 | 85 | −17.9 |
| | 2 | 0 | 0 |
| | 2 | 55 | 11.4 |
| | 2 | 85 | 14.8 |
| | 3 | 0 | 0 |
| | 3 | 55 | 14.9 |
| | 3 | 85 | 18.9 |
| Placebo | 1 | 0 | 0 |
| | 1 | 55 | −10.5 |
| | 1 | 85 | −0.4 |
| | 2 | 0 | 0 |
| | 2 | 55 | 89.7 |
| | 2 | 85 | 144.8 |
| | 3 | 0 | 0 |
| | 3 | 55 | 19.7 |
| | 3 | 85 | 27.7 |

TABLE 6
HEART RATE ASSESSMENT

| Test Article | Day | Time of Exercise | Heart Rate |
|---|---|---|---|
| Ribose | 1 | 9 | 155 |
| | 1 | 17 | 158 |
| | 1 | 25 | 161 |
| | 2 | 9 | 154 |
| | 2 | 17 | 153 |
| | 2 | 25 | 156 |
| | 3 | 9 | 148 |
| | 3 | 17 | 153 |
| | 3 | 25 | 157 |
| Placebo | 1 | 9 | 158 |
| | 1 | 17 | 161 |
| | 1 | 25 | 169 |
| | 2 | 9 | 163 |
| | 2 | 17 | 168 |
| | 2 | 25 | 173 |
| | 3 | 9 | 158 |
| | 3 | 17 | 169 |
| | 3 | 25 | 172 |
